(19) United States
(12) Patent Application Publication
(10) Pub. No.: US 2006/0270571 A1
(43) Pub. Date: Nov. 30, 2006

(54) Deactivation of mineral encapsulated nanobacteria

(76) Inventors: Peter A. Burke, Concord, OH (US); Gerald E. McDonnell, Hampshire (GB); Kathleen A. Fix, Willoughby, OH (US)

Correspondence Address: HUDAK, SHUNK & FARINE, CO., L.P.A., 2020 FRONT STREET, SUITE 307, CUYAHOGA FALLS, OH, 44221, US

(21) Appl. No.: 11/138,138

(22) Filed: May 26, 2005

(51) Int. Cl. A61K 8/00

(52) U.S. Cl. 510/130

(57) ABSTRACT

Compositions and methods for deactivating articles contaminated with nanobacteria, generally comprise a dispersant and/or a dissolution agent, and a deactivator. The methods and compositions of the invention are advantageously utilized to decontaminate and/or sterilize various articles such as medical and manufacturing devices or surfaces.

### FIELD OF THE INVENTION

The invention relates to compositions and methods for deactivating articles contaminated or potentially contaminated with nanobacteria which generally have a mineral-containing outer protective layer.

### BACKGROUND OF THE INVENTION

Nanobacteria are thought to contain various organisms dramatically smaller than previously identified bacteria and have a size on the order of 500 nanometers or less. They are characterized by a protective layer, e.g., occluded or encapsulated as by a cell wall or membrane, an inorganic shell, etc., which often contains calcium salts, e.g., apatite, which becomes thicker with age. Such nanobacteria have been linked or associated with various calcification-related diseases in humans including stone formation (kidney and gallstones), malacoplakia, atherosclerosis, heart valve deposits and related to various carcinomas. Nanobacteria have created some concern in healthcare and pharmaceutical industries regarding the confirmation of infectivity and how the risks of transmission can be limited.

Nanobacteria are generally thought to be very difficult to deactivate inasmuch as at least one study Extraordinary Survival of Nanobacteria Under Extreme Conditions, Proc. SPIE Int. Soc. Opt. Eng. 3441, M. Bjorklund, N. Ciftcioglu and E. O. Kajander, 1998, pp 123-129 has found that they are not deactivated by physical or chemical treatments including autoclaving (20 minutes at 121° C.), UV treatment (1 to 3 hours), microwave heating (boiling samples 5 times), and various biocides. The biocides include ethanol, glutaraldehyde, formaldehyde, hypochlorite, hydrogen peroxide, hydrochloric acid, sodium hydroxide, guanidium hydrochloride, urea, Erifenol (100% product contains 50% potassium persulfate, 5% sulfaminoic acid), Klorilli (100% contains sodium N-chloro-p-toluenesulfonamide-3-hydrate and 20000 ppm active chlorine), and the like.

U.S. Pat. No. 6,706,290, the inventor of which is the same as the author of the immediately above Extraordinary Survival of Nanobacteria Under Extreme Conditions article relates to providing methods of treating patients infected with nanobacteria. In particular, U.S. Pat. No. 6,706,290 provides a method for reportedly preventing the recurrence of kidney stones in a patient that has suffered from kidney stones, comprising administration of an antibiotic, a bisphosphonate, or a calcium chelator, either alone or in combination, in an amount effective to inhibit or prevent the growth and development of nanobacteria.

It is further stated in U.S. Pat. No. 6,706,290, in this and the following paragraphs, that nanobacteria approach the theoretical limit of the self-replicating life with a size of only one one-hundredth of-that of usual bacteria. Nanobacteria can be isolated from mammalian blood and blood products (see, U.S. Pat. No. 5,135,851 to Kajander, the contents of which are incorporated herein by reference). Energy-dispersive X-ray microanalysis and chemical analysis reveals that nanobacteria produce biogenic apatite on their cell envelope. The thickness of the apatite depends mostly on the culture conditions of the nanobacteria. Nanobacteria are the smallest cell walled, apatite forming bacteria isolated from mammalian blood and blood products. Their small size (0.05-0.5 μm), and unique properties make their detection difficult with conventional microbiological methods. In nanobacteria-infected mammalian cells, electron microscopy revealed intra- and extracellular acicular crystal deposits, stainable with von Kossa staining and resembling calcospherules found in pathological calcification.

Competition for nutrients necessary for life is enormous in natural environments and thus clever adaptations and survival strategies for unfavorable conditions are needed. Bacteria can form spores, cysts and biofilm, which help them survive unfavorable periods of time. Bacteria in such forms have significantly slower metabolic functions, but vegetative cells can slow down their metabolism as well. The increased resistance of bacteria in biofilm or as spores is not only because of the slower metabolic rate. The impermeable structures around the organism serve as mechanical barriers blocking the entrance of potentially harmful compounds. Some additional mechanisms are also known which help in the survival of bacteria. The heat resistance of bacterial spores can be attributed to three main factors; these are protoplast dehydration, mineralization and thermal adaptation. Radiation resistance is commonly associated with sophisticated DNA repair systems. Multiplication and minimizing metabolic rate are obviously the main preconditions for bacterial survival, allowing time for the repair of DNA and other damaged cellular components. Very slow metabolism and ability to form biofilm are also characteristics of nanobacteria. Because of their minimal size, the presence of complicated systems for nucleic acid repair in nanobacteria seems unlikely.

Apatite may play a key role-in the formation of kidney stones. The crystalline components of urinary tract stones are calcium oxalate, calcium phosphate, struvite, purines, or cystine. The majority of urinary stones are admixtures of two or more components, with the primary admixture being calcium oxalate and apatite. Furthermore, fermenter model studies have shown that calcium phosphate are always formed initially, and may subsequently become coated with calcium oxalate or other components. Urinary tract infection, causing struvite and carbonate apatite formation, is a common cause of kidney stones. Conventional therapy has usually consisted of surgical removal of the stone, combined with a short course of antimicrobial therapy. Such treatment is curative in about 50% of cases. Recurrent stone formation and progressive pyelonephritis occur in those who are not cured. The morbidity and expense that result from this disease is significant.

Tissue calcification of carbonate apatite in nature is common in other diseases, e.g., atherosclerotic plaques accumulate calcium phosphate. 25% of atherosclerotic plaques in human aorta specimens were found to contain nanobacteria by immunoassay and immunohistochemical staining. Hemodialysis patients can develop extensive metastatic and tumoral calcification. Acute periarthritis is apatite arthropathy related to intratendinous calcifications. Apatite crystals also cause inflammation when injected into the synovial space. Tissue calcification is also found in several kinds of cancer.

Pulp stones or denticles are polymorphous mineralized bodies of various sizes occasionally found in the pulpal connective tissue of human teeth. Their etiology remains unclear although they have been frequently associated with aging or pathology of the pulp. They may also be present in permanent teeth. Although pulp stones have been extensively studied morphologically, their origin is still obscure and little is known about their chemical composition. A histochemical study of pulpal calcifications has shown that the organic matrix consists of reticular connective tissue fibers and a ground substance containing glycoproteins and acid polysaccharides. The mineral phase of pulp calcification has been studied with X-ray energy dispersive spectrometry and chemical analysis and has proven that calcium salts are deposited in the form of apatite, possibly carbonate apatite. In fact, there is not much difference between the chemical structure of a tooth and denticles. Bone and tooth formation in the body have similar mechanisms, leaving many unanswered questions. Apatite formation in the body (except in tooth and bone) is called pathologic biomineralization, e.g., dental pulp stones, kidney stones, and joint calcifications.

Malacoplakia is a rare chronic inflammatory disease of unknown cause, but a bacterial factor has been strongly implicated. It may be fatal. The disease is characterized by von Kossa staining positive, calcified laminated or target-shaped bodies termed Michaelis-Gutmann bodies which are composed of apatite. The structure of these calcospherules closely resembles calcified nanobacteria.

Tissue calcifications are found in several diseases such as ovarian serous tumor, papillary adenocarcinoma of the endometrium, breast carcinoma, papillary carcinoma of the thyroid, duodenal carcinoid tumor, and craniopharyngioma. In many malignant tumors, needle-shaped crystals are found in epithelial cells. To detect this kind of calcification it is necessary to use electron microscopy, since the crystals are too small to be seen with the light microscope, and their origin is unknown. Many malignant cells have receptors for nanobacterial adherence. They could introduce nanobacteria into the tumor with subsequent calcification. Furthermore, some dividing cells under inflammatory stimuli may have receptors for adherence, e.g., in atherosclerotic plaques known to have calcium phosphate accumulation. In this disease, although electron probe analysis showed that the surface and interior of the mineral deposit had the same chemical composition, SEM revealed different kinds of structures such as spherical particles and fibers that resemble nanobacteria. Similarly, acute periarthritis has been associated with the presence of hydroxyapatite crystals in the joints.

Alzheimer plaques may be labeled with anti-nanobacterial polyclonal antibodies. These polyclonal antibodies contain some autoantibodies, and the inventors of U.S. Pat. No. 6,706,290 have reportedly also obtained some monoclonal autoantibodies in nanobacterial immunizations. Slow bacterial infection has been suggested to play a role in autoimmune diseases. Tissue calcification is often present in these diseases. Nanobacteria are a new example of slowly growing organisms, infecting man for long periods of time. The apatite structure and anomalous nucleic acids may contribute to abnormalities in immune response to this infection.

Several aspects of biogenic apatite nucleation, crystal growth and morphology have been determined both in vivo and in vitro. However, many details remain unresolved, including the specific nature of the initial precipitating phases, the mechanism and factors that control the incorporation of ionic impurities into the crystal lattice, details of the crystallographic ultrastructure and morphology in mineralized tissues (bone, dentine), and the relationship of the inorganic components with the complex collagen based matrix. The reason behind the calcium phosphate deposition in many diseases remains speculative. It has been shown that an accumulation of calcium in mitochondria, which is presumably dependent upon residual substrate for energy production, appeared to cause calcification. Amorphous calcium phosphate in the form of spheroids, and possibly fine fibrils and granules, also appears to play a role in calcification by their transformation into apatite.

WO 03/030949 relates to methods such as radiation for reportedly sterilizing biological materials to reduce the level of one or more biological contaminants or pathogens therein, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs.

### SUMMARY OF THE INVENTION

Compositions and methods are disclosed for deactivating articles such as medical devices, manufactured items, and the like, and surfaces thereof contaminated with nanobacteria which generally have a mineral-containing outer protective layer. While the layer may generally be a cell wall or membrane which occludes or encapsulates the bacteria, it is generally thought to be an occluding or encapsulating inorganic shell such as a calcium containing mineral. The compositions of the present invention which deactivate the nanobacteria generally comprise a dispersant and/or a dissolution agent, and a deactivating agent. While not fully understood, it is thought that the dispersant will generally break up or emulsify the nanobacteria, which is generally suspected as existing in clusters or matrices, and that the dissolution agents will generally dissolve or break apart the protective layer thereby exposing the bacteria for deactivation by the deactivating agent. Thus, in one embodiment, a composition which renders the nanobacteria innocuous comprises a dissolution agent and a deactivating agent. In another embodiment, the composition comprises a dispersant agent and a deactivating agent while in yet another embodiment the composition comprises a dispersant, a dissolution agent, and a deactivating agent.

A nanobacteria deactivation composition, comprising: a dispersant comprising a hydrophilic polymeric dispersant; or an anionic wetting agent, a nonionic wetting agent, a cationic wetting agent, or an amphoteric wetting agent, or combinations thereof, said anionic wetting agent being free of a sodium organo sulfate and a sodium aliphatic-aryl sulfonate, and said cationic wetting agent being free of a quaternary aliphatic-aryl ammonium chloride; and a deactivating agent.

A nanobacteria deactivation composition, comprising: a dissolution agent comprising a nitrogen-free organic acid having at least one carboxylic acid group and a total of from 2 to about 20 carbon atoms, a phosphoric acid containing compound, a sulfonated polyphosphoric acid compound, a polyphosphonate having three or more phosphonate groups, an enzyme; or salts thereof; or combinations thereof; said dissolution agent being free of an organic acid having from three to about five carboxylic acid groups; and a deactivating agent; and optionally a dispersant as set forth in the preceding paragraph.

### DETAILED DESCRIPTION OF THE INVENTION

Articles including surfaces thereof may contain unwanted or potentially dangerous nanobacteria. It is important that such nanobacteria be destroyed, eradicated, or otherwise deactivated in order to prevent harm or minimize damage to a human, etc.

Nanobacteria are generally nano-sized organisms that have a protective coating such as an occluded or encapsulated cell wall or membrane or more likely an inorganic shell, etc. The size of the nanobacteria as determined by electron microscopy is generally less than about 500 nanometers, and often from about 0.5 to about 300 or from about 20 to about 200 nanometers in diameter. Thus, the average size of nanobacteria is generally a small fraction such as about one-hundredth of typical bacteria. Nanobacteria have only been recently discovered and the exact type of organism is not fully understood but one such species thereof is thought to be N. sanguineun.

Studies have revealed that nanobacteria produce a biogenic protective layer which generally encapsulates, forms an inorganic shell or otherwise occludes the nanobacteria and serves as a protective barrier that blocks entrance of compounds that can deactivate the nanobacteria. The protective layer, such as an inorganic shell, is very resistant to conventional compositions and methods used to deactivate typical microorganisms without such coatings. The protective shell, etc., generally contains calcium, typically in significant amounts. A specific type of shell material are the various types of apatite compounds such as those represented by, but not limited to, the formula Ca₅(PO₄)₃X where X is a halide such as fluoride or chloride, OH, or Ca₅([PO₄9 [CO₃])₃Cl aka carbonate apatite), or the like. Hydroxyapatite is the mineral that also makes up the teeth and bones in all vertebrate animals. Alternatively, the protective layer can be some other mineral containing a calcium salt, such as calcium carbonate. The thickness of the protective layer can vary.

While not fully understood, it is believed that the nanobacteria replicate and spread as follows. Referring to FIG. 1, a nanobacterium attaches to a surface, grows, and replicates forming clusters or matrices comprising a colony of nanobacteria generally connected by their protective layer. As growth continues, sections of the clusters or matrices can break off and bind to other surfaces. Such biogenic growth can readily occur on various articles and surfaces thereof. Nanobacteria appear to grow at a much slower rate than typical bacteria.

Various compositions and methods for utilizing the same are disclosed for deactivating articles and surfaces thereof contaminated or potentially contaminated with nanobacteria. By the term “nanobacteria deactivation composition” it is meant a composition containing a dispersant and/or a dissolution agent, and a deactivating agent which generally exist as an aqueous composition in either a concentration form, or in amounts indicated herein that can be applied to various articles or surfaces thereof contaminated or potentially contaminated with nanobacteria. Such articles include, general medical devices including surgical instruments, telescopes, cameras, and the like; medical aid devices such as syringes, tubing, catheters, and the like; medical lumen devices such as endoscopes, and the like; various medical mortuary items; various dental devices; various tattooing/piercing equipment; various operating theater equipment/surfaces; and various veterinary equipment. Other articles include manufactured devices such as pharmaceutical items as for example vaccines, saline solutions, drug delivery solutions, and the like.

As noted, the compositions of the present invention include one or more dispersants and/or one or more dissolution agents, along with at least one deactivating agent although at times only the deactivating agents are necessary. It is desirable that the one or more dispersants, dissolution agents, and deactivation agents are compatible with one another when in a solution form such that there is no or little precipitation. The different components of the nanobacterial deactivation compositions of the present invention are utilized in suitable amounts to deactivate the nanobacteria by one or more routes, for example the clustering of groups to the bacteria, or dissolution thereof, or both. It is to be understood that the amounts of the one or more compounds of the following classes of the general categories of dispersants, dissolution agents, and deactivators can vary greatly depending upon the type of nanobacterial contamination or potential contamination of an article or surface thereof. Thus, depending upon the end use, the amount of the at least one deactivation agent can be large or small, and the same is true with regard to the at least one dispersant compound and/or dissolution agents.

The one or more dispersants generally include various polymers or various surfactants such as wetting agents that include anionic wetting agents, nonionic wetting agents, cationic wetting agents, and amphoteric wetting agents, or combinations thereof.

The polymeric dispersants are generally hydrophilic and have a number average molecular weight of from about 3,000 or about 5,000 to about 8,000, or about 10,000, or even about 15,000. Polymeric dispersants are known to the literature and to the art and include various polyacrylates and various polymethacrylates wherein the ester portion contains from about 2 to about 10 carbon atoms such as methyl, ethyl, butyl, and 2-ethylhexyl polyacrylate, and various polyacrylamides such as methacrylamide and ethacrylamide, various polymaleates, various polyalkylene oxides such as polymethylene oxide and polyethylene oxide, various polyphosphates, various polyphosphate esters, AMPS® (acrylamidomethylpropylsulfonic acid) polymers, various polysulfonates, various polysilicates, and the like. Such polymers are commercially available under the trade names of Acusol®, Acumer®, Tamol®, Alkanol®, Goodrite®, and Versa®. Suitable dispersants also include copolymers derived from two or more monomers utilized to make the above polymers or with other monomers such as maleic anhydride-sulfonated styrene copolymers, acrylic anhydride-acrylamide copolymers, acrylic anhydride-sulfonated acrylamide copolymers, acrylic anhydride-AMPS copolymers, and copolymers derived from combinations of acrylic acid, acrylate, methacrylate, acrylamide, or alkylene oxide monomers. An example of a terpolymer is one that is derived from acrylic anhydride, maleic anhydride, and AMPS®. While polymers derived from four or more monomers such as quadpolymers can be utilized, they generally tend to be expensive and less effective due to the dilution effects of the individual monomers.

Various dispersants also include numerous surfactants that broadly can be classified as anionic wetting agents, nonionic wetting agents, and cationic wetting agents. Literally thousands of such wetting agents exist and a list of such compounds that can be utilized is too numerous to set forth. However, the guiding principle is that they are compatible with the dissolution agents and the deactivating agents in that they generally do not precipitate out of solution and are effective in suspending, emulsifying, or otherwise breaking up the clusters and matrices of the nanobacteria. Moreover, some of the surfactants can also serve as dissolution agents.

Anionic surfactants such as anionic wetting agents generally include various ether sulfates; various non-sodium organo sulfates; various organo phosphates; various sulfoacetates; various sulfonates including various metal (non-aliphatic) aryl organo sulfonates and various metal (non-aryl) aliphatic organo sulfonates, various amine sulfonates, and various organo sulfonic acids; and various sulfosuccinates; or salts thereof.

Examples of various ether sulfates that contain aliphatic and/or aromatic groups or both having a total of from about 8 to 50 carbon atoms include ammonium ether sulfate, sodium tridecyl ether sulfate, sodium trideceth sulfate, ammonium lauryl ether sulfate, sodium lauryl ether sulfate, ammonium lauryl ether sulfate, sodium nonylphenol ether sulfate, alkyl phenol ether sulfate, sodium ether sulfate, and combinations thereof.

Non-sodium organo sulfates that contain aliphatic and/or aromatic groups or both having a total of from about 8 to 40 carbon atoms include e.g., potassium lauryl sulfate, potassium decyl sulfate, potassium octylphenol ethoxylated sulfate, potassium nonylphenol ethoxylated sulfate, ammonium nonylphenol ethoxylated sulfate, potassium 2-ethyl-hexyl sulfate, potassium octyl sulfate, ammonium lauryl sulfate, ammonium laureth sulfate, potassium laureth sulfate, magnesium lauryl sulfate, TEA lauryl sulfate, amine organo sulfates and combinations thereof.

Organo phosphates that contain aliphatic and/or aromatic groups or both having a total of from about 8 to 40 carbon atoms include e.g., sodium lauryl phosphate, sodium decyl phosphate, sodium octylphenol ethoxylated phosphate, sodium nonylphenol ethoxylated phosphate, ammonium nonylphenol ethoxylated phosphate, sodium 2-ethyl-hexyl phosphate, sodium octyl phosphate, ammonium lauryl phosphate, ammonium laureth phosphate, sodium laureth phosphate, magnesium lauryl phosphate, TEA lauryl phosphate, and combinations thereof.

Examples of a sulfoacetate anionic surfactant that contain aliphatic and/or aromatic groups or both having a total of from about 8 to 20 carbon atoms include sodium lauryl sulfoacetate.

Examples of alkali metals (e.g. sodium, potassium) aliphatic (non-aryl) organo sulfonates that contain an aliphatic or dialiphatic group independently having from 8 to about 20 carbon atoms include sodium alkane sulfonate such as sodium octane sulfonate, sodium olefin sulfonate such as sodium C14-16 olefin sulfonate, and sodium alpha olefin sulfonate, and combinations thereof. Examples of amine-containing sulfonates that contain aliphatic and/or aromatic groups or both having a total of from about 8 to 40 carbon atoms include isopropylamine alkylbenzene sulfonate, TEA-dodecylbenzene sulfonate, TEA-alkyl benzene sulfonate, amine alkyl aryl sulfonate, and isopropyl amine dodecylbenzene sulfonate, and combinations thereof. Anionic organo sulfonic acids that contain aliphatic or aromatic groups and/or both having a total of from about 6 to about 20 carbon atoms include alkyl benzene sulfonic acid, toluene sulfonic acid, and the like.

Examples of anionic sulfosuccinate wetting agents having a total of from about 8 to about 40 carbon atoms include disodium alkyl ether sulfosuccinate, disodium oleamido MIPA sulfosuccinate, disodium laureth sulfosuccinate, and sodium dioctylsulfosuccinate, and combinations thereof.

Sodium aliphatic sulfates are not very soluble and thus are avoided, i.e. none used, as are alkali as metal aliphatic-aryl sulfonates per se and if utilized, exist in only very small amounts thereof, such as 1,000 or 750 parts by weight or less and desirably 500 parts by weight or less per 1,000,000 parts by weight of the nanobacteria deactivation composition.

The nonionic surfactant wetting agents include various alkoxylates, various amides, various esters, various ethoxylates, various triglycerides, and the like. Such organo wetting agents generally have a total of from about 1 or about 5 to about 20 or about 50 carbon atoms, except for the polymers that have substantially higher numbers of carbon atoms. Moreover, other nonionic surfactants can generally be utilized so long as they are compatible with other components such as other wetting agents, various dissolution agents, and the various deactivators.

Examples of alkoxylates that contain aliphatic or aromatic groups or both include various polyaliphatic and/or aromatic alkoxylates, various polyalkoxylated amides, various alkylphenol alkoxylates, various alkylphenol block copolymers, various polyalkylene oxide block copolymers, various alcohol alkoxylates, and various butyl based block copolymers, or their salts and combinations thereof.

The various amide nonionic wetting agents that contain aliphatic or aromatic groups or both include various fatty alkanolamides, various modified fatty alkanolamides, various monoethanol amides and dimethanol amides, oleyl diethanolamide, lauryl diethanolamide, coconut diethanolamide, coco diethanolamide, lauramide DEA, fatty diethanolamide, PEG-6 cocamide, lauramide MEA, Cocamide DEA, coco monoethanolamide, PEG-6 lauramide, coco monoisopropanolamide, Cocamide MIPA, Cocamide MEA, or their salts and combinations thereof.

Numerous nonionic ester wetting agents that contain aliphatic or aromatic groups or both exist as known to the art and to the literature and examples thereof include various phosphate esters such as various alkyl ether phosphates, various alcohol ethoxylated phosphate esters and various tridecyl alcohol phosphate esters wherein the alcohol portion is from 1 to about 20 carbon atoms such as nonylphenol ethoxylated phosphate ester and oleyl alcohol ethoxylated phosphate ester, and the like. Other esters include cetyl palmitate, methyl laurate, methyl palitate/oleate, glycol stearate (and) stearamide AMP, glyceryl stearate (and) PEG 100 stearate, isopropyl palmitate, PEG-4 dioleate, PEG-12 laurate, propylene glycol stearate, sorbitol esters, ethoxylated sorbitol esters, PEG-2 stearate, various glycols having from 2 to 8 carbon atoms, glycol distearate, glycol stearate, glyceryl dilaurate, glyceryl laurate, glyceryl oleate, ethylhexyl palmitate, PEG-4 dilaurate, PEG-8 dilaurate, PEG-8 distearate, PEG-8 oleate, PEG-12 dilaurate, PEG-12 dioleate, PEG-12 distearate, PEG-150 distearate, PEG-150 stearate, nonylphenol POE-10 phosphate ester, nonylphenol POE-6 phosphate ester, nonylphenol POE-4 phosphate ester, nonylphenol POE 8 phosphate ester, nonylphenol POE-12 phosphate ester, PEG-400 diolate, various sorbitol esters, various ethoxylated sorbitol esters, and various polysorbates such as polysorbate 20, polysorbate 60, polysorbate 80, and polysorbate 85, or combinations thereof. Such esters are commercially available under the trade names of Triton X® compounds and Tween® compounds.

Various ethoxylate nonionic wetting agents that contain aliphatic or aromatic groups or both include alkylphenol ethoxylate, fatty alkyl ethoxylate, alcohol ethoxylate, tallow amine ethoxylate, the various oleyl alcohol ethoxylates, the various stearic acid ethoxylates, the various octyl phenol ethoxylates, the various nonyl phenol ethoxylate, decyl alcohol ethoxylate, tridecyl alcohol ethoxylate, lauryl alcohol ethoxylate, castor oil ethoxylate, sorbital trioleate ethoxylate, sorbital monooleate ethoxylate, tallow amine ethoxylate, and combinations thereof.

The various triglyceride nonionic wetting agents that contain aliphatic or aromatic groups or both include caprylic/capric triglyceride, caprylic triglyceride, tri caprylic/capric triglyceride ester, hydrogenated vegetable oil, and combinations thereof.

The cationic wetting agents include numerous compounds known to the literature and to the art. Generally any cationic wetting agent can be utilized so long as it is generally compatible with any other one or more dispersants, dissolution agents, and the various deactivating agents. Thus, the following is representative of various suitable cationic wetting agents.

A desired class of cationic agents is one or more aliphatic (non-aryl) ammonium halides, carbonates or sulfates that contain from 1 to about 4 aliphatic groups, preferably alkyl groups, independently, having from 1 to about 30 carbon atoms, and wherein the halide is chloride, bromide, or iodine. Examples of such aliphatic quaternary compounds include tetrabutyl ammonium halide, tetraethyl ammonium halide, tetra propyl ammonium halide, tetra methyl ammonium halide, tetra octyl ammonium halide, butyl triethyl ammonium halide, methyl trioctyl ammonium halide, methyl tricapryl ammonium halide, methyl tributyl ammonium halide, myristyl trimethyl ammonium halide, cetyl trimethyl ammonium halide, tetradecyl trimethyl ammonium halide, hexadecyl trimethyl ammonium halide, lauryl trimethyl ammonium halide, dodecyl trimethyl ammonium halide, phenyl trimethyl ammonium halide, dimethyl hydroxypropylammonium chloride polymer, various dialkyl dimethyl ammonium halides; dialkyl(C8-10) dimethyl ammonium halide, decyl isononyl dimethyl ammonium halide, didecyl dimethyl ammonium halide, dioctyl dimethyl ammonium halide, ditallow dimethyl ammonium halide, methyl bis(tallowamido ethyl)-2-hydroxyethyl ammonium methyl sulfate, methyl bis(tallowamido ethyl)-2-hydroxyethyl, methyl bis(soya amidoethyl)-2-hydroxylethyl ammonium methyl sulfate, methyl bis(canola amidoethyl)-2-hydroxythyl ammonium methyl sulfate, methyl bis(tallowamido ethyl)-2-tallow imidazolinium methyl sulfate, and methyl bis(ethyl tallowate)-2-hydroxyethyl ammonium methyl sulfate, or combinations thereof.

Another class of cationic wetting agents is various one or more aryl ammonium halides (non-chloride) or aliphatic aryl ammonium halides (non-chloride), carbonates, or sulfates wherein when an aliphatic group exists, the number thereof can be from 1 to about 4, independently, containing from about 1 to about 30 carbon atoms with alkyls being preferred, and the number of aryl groups is from 1 to about 4 such groups, independently, containing from 6 to about 30 carbon atoms with the halide being either bromide or iodide. Naturally, the total number of alkyl and/or aryl groups is 4. Examples of such aryl (non-chloride) or aliphatic aryl (non-chloride) quaternary compounds include benzyl trimethyl ammonium bromide, benzyl tributyl ammonium bromide, lauryl dimethyl benzyl ammonium bromide, cetyl dimethyl benzyl ammonium bromide, dodecyl dimethyl benzyl ammonium bromide, tetradecyl dimethyl benzyl ammonium bromide, hexadecyl dimethyl benzyl ammonium bromide, octadecyl dimethyl benzyl ammonium bromide, dodecyl dimethyl benzyl ammonium iodide, tetradecyl dimethyl benzyl ammonium iodide, hexadecyl dimethyl benzyl ammonium iodide, octadecyl dimethyl benzyl ammonium iodide, dodecyl trimethyl benzyl ammonium iodide, and also dodecyl dimethyl ammonium carbonate, dodecyl dimethyl ammonium bicarbonate, and combinations thereof.

The quaternary compounds of the present invention are generally free of any sodium aliphatic aryl ammonium chloride compound. Thus, if utilized, only very small amounts thereof such as 5,000 parts by weight or less, or 1,000 parts by weight or less, and desirably 500 parts by weight or less per 1,000,000 parts by weight of the nanobacteria deactivation composition.

When any of the cationic quaternary compounds of the present invention are utilized, the use of anionic wetting agents are generally avoided since they interact therewith and often result in precipitation that negates the activity of both wetting agents.

Various quaternary phosphonium compounds can also be utilized wherein the number of aliphatic groups can be from 1 to 4 and the number of aryl groups can be 1 to 4 with the proviso that the total number of such aliphatic and/or aryl groups is 4, wherein each aliphatic group (preferably alkyl) group, independently, contains from 1 to about 30 carbon atoms and each aliphatic-aryl group contains from 6 to about 30 carbon atoms. The halide can be a chloride, bromide, or iodide. Examples of suitable quaternary phosphonium compounds include ethyl triphenyl phosphonium halides, butyl triphenyl phosphonium halides, benzyl triphenyl phosphonium halides, methyl triphenyl phosphonium halides, tetraphenyl phosphonium halides, tributyl tetradecyl phosphonium halides, and combinations thereof.

Still another class of wetting agents or surfactants include various amphoteric compounds including betaines and sultaines, having from 2 to about 20 carbon atoms, and the like, such as cocamidopropyl betaine, cocoamidopropyl betaine, lauryl betaine, hydrogenated cocamidopropyl betaine, laurylamidopropyl betaine, cocamidopropyl hydroxysultaine, or combinations thereof. Other amphoteric compounds are based upon dodecyl-p-aminobutyric acid, dodecyl-di(aminoethyl)-glycine, or an imidazol ring and are commercially available as Armeen®, Tego® or Miranol®.

The total amount of the one or more dispersants, when utilized, is generally from about 100 to about 100,000 or about 50,000 or about 10,000 parts by weight, and desirably from about 500 to about 5,000 parts by weight per 1,000,000 parts by weight of the nanobacteria deactivation composition.

The one or more dissolution agents, as noted, generally serve to at least partially dissolve the nanobacteria protective coating that is generally calcified. Suitable dissolution agents include various organic salts, various organic acids often containing phosphorus, or salts thereof, and the like. Such compounds are thought to remove or reduce the protective layer occluding or encapsulating the bacteria. Organic solvents are avoided, i.e. none used, since they do not dissolve the calcium-containing protective layer of the nanobacteria. Organic solvents include various hydrocarbons containing from 6 to about 20 or about 30 carbon atoms and include aliphatic, aromatic, or combinations thereof such as hexane, heptane, octane, decane, ect., benzene, toluene, xylene, and the like. If utilized, they are utilized in very small amounts such as 1,000 parts or less, desirably 750 parts or less, and preferably 500 parts by weight or less per 1,000,000 parts by weight of the total nanobacteria deactivation composition.

One group of organic acids are the various nitrogen free carboxylic acids having a total of from 2 to 20 carbon atoms having only one or two acid groups and one or more hydroxyl groups include tartaric acid, gluconic acid, glycolic acid, hydroxysuccinic acid, galactaric acid, hydroxypropionic acid, lactic acid, glyceric acid, hydroxybutyric acid, hydroxyisobutyric acid, hydroxy methylbutyric acid, bis(hydroxymethyl) propionic acid, gibberellic acid, hydroxyoctadecanoic acid, di-tert-butyl hydroxybenzoic acid, benzilic acid, hydroxyl fluorenecarboxylic acid, hydroxydecanoic acid, hydroxynaphthalenecarboxylic acid, hydroxybenzenedicarboxylic acid, hydroxymethylbenzoic acid, hydroxyphenylacetic acid, mandelic acid, hydroxymethoxybenzoic acid, methoxysalicylic acid, hydroxyoctanoic acid, hydroxycinnamic acid, dihydroxycinnamic acid, dihydroxy-hydrocinnamic acid, hydroxyphenylpropionic acid, dihydroxytartaric acid, hydroxymethoxycinnamic acid, chlorohydroxybenzoic acid, chloromandelic acid, chloro phthalic acid, salicylic acid, chlorosalicylic acid, citrazinic acid, dibromo hydroxybenzoic acid, dichlorohydroxy-benzoic acid, dichlorosalicylic acid, galactouronic acid, glucuronic acid, hydroxypropanedioic acid, hydroxyphenyl propionic acid, lactic acid, methoxysalicylic acid, trihydroxybenzoic acid, or their partial salts and combinations thereof.

Another group of organic acids are various hydroxyl free and nitrogen free saturated or unsaturated dicarboxylic acids having from 2 to about 20 carbon atoms and can contain nitrogen atoms. Examples include oxalic acid, malonic acid, succinic acid, glutaric acid, adipic acid, pimelic acid, maleic acid, fumaric acid, decanedoic acid, camphoric acid, benzenedicarboxylic acid, phthalic acid, cyclohexanedicarboxylic acid, cyclohexanediacetic acid, octanedioc acid, homophthalic acid, phenylmalonic acid, cyclopentanediacetic acid, nonanedioic acid, benzylmalonic acid, phenylenediacetic acid, phenylsuccinic acid, bromosuccinic acid, carboxyphenacetic acid, cyclobutanedicarboxylic acid, cyclohexanedicarboxylic acid, decanedicarboxylic acid, dibromosuccinic acid, dichlorophthalic acid, diethylmalonic acid, diglycolic acid, dimethylmalonic acid, dimethyl pentanedioic acid, dimethylsuccinic acid, ethylmalonic acid, glutamic acid, hexenedioic acid, imino diacetic acid, methylmalonic acid, methylsuccinic acid, naphthalene dicarboxylic acid, oxalacetic acid, oxopentanedioic acid, undecane dicarboxylic acid, dipicolinic acid, or their partial salts, and combinations thereof.

Polymeric acids or phosphorus-containing acids can also be utilized such as phosphinocarboxylic acid, e.g. Belsperse® 161 or 164, sulfonated phosphinocarboxylic acid, e.g., Belclene® 400, nitrilotriacetic acid, polymaleic acid, polyacrylic acid, or their partial salts and combinations thereof. Polymeric acids have at least 5 or 10 acid repeat units and are thus not considered to be carboxylic acids.

Compounds containing three or more phosphonate groups such as the various polyphosphonates can be utilized including ATMP, DETA phosphonate, BHMT phosphonate, EDT phosphonate, hexam-ethylenediaminetetra(methylenephosphonic acid), HMDT phosphonate or their partial salts and combinations thereof that are commercially available as Dequest®, Unihib®, Mayoquest® and Briquest®. Disphosphonates are generally not used inasmuch as they are generally not effective dissolution agents and hence the compositions of the present invention are free thereof. That is, if utilized, they exist in very small amounts such as 1,000 parts or less, and desirably from 750 parts or less or 500 parts or less by weight per 1,000,000 parts by weight of the nanobacteria deactivation composition.

Other dissolution agents include phosphate esters such as pyrophosphate, tripolyphosphate, hexametaphosphate, tridecyl alcohol phosphate ester, nonylphenol ethoxylate phosphate ester, nonylphenol POE n phosphate ester, phosphate esters of an alkyl polyethoxyethanol, or their partial salts and combinations thereof.

Still another class of dissolution agents are various enzymes including proteases such as amylases, lipases, and various phosphates, or other digestive enzymes, and combinations thereof.

Generally, organic acids having 3 to about 5 or more carboxyl groups and optionally containing 1 or more hydroxyl groups and/or optionally containing one or more nitrogen atoms to about 5 are avoided as dissolution agents. Accordingly, no hydroxyl-containing organic acids having 3 to about 5 carboxylic acid groups, etc. are utilized, and if utilized, only in very small amounts such as 1,000 parts by weight or less, desirably 750 parts by weight or less, preferably 500 parts by weight or less per 1,000,000 parts by weight of the nanobacteria deactivation composition.

The various dissolution agents are generally utilized under alkaline pH conditions and thus desirably have a pKa less than the pH of the solution. In other words, the dissolution compound should be partially or fully de-protonated for maximum effect.

In conjunction with the above one or more different types of dissolution agents, the nanobacteria can be subjected to various physical treatments either before or after utilization of the compositions of the present invention. Such treatments include autoclaving at temperatures of from about 110° C. to about 140° C. from about 3 to about 30 minutes; ultraviolet radiation of from about 1 to about 1,000 watts having a wavelength of from about 100 to about 3,900° A for a time of from about 1 hour to about 1 or 2 days. Heating can also be utilized at temperatures of about 45° C. to about 90° C. for a time from about 2 to about 30 minutes. Another treatment involves sonication of the liquid in which the nanobacteria are entrained.

An amount of the dissolution agent is utilized to effectively remove the protective layer from the nanobacteria and yet should not be in an excess amount that generally would attack the article, for example, various metal devices or manufactured devices, to which the nanobacteria are attached. The amount of the one or more dissolution agents is generally from about 5 to about 100,000 or 50,000 or 10,000 parts by weight, and desirably from about 200 to about 1,000 parts by weight per 1,000,000 parts by weight of the nanobacteria dissolution composition.

The one or more deactivation agents are utilized to deactivate, for example, to disinfect, sterilize, sanitize or generally to render innocuous the nanobacteria. The bacteria can generally be deactivated once they have been exposed. Exposure will occur when the protective layer of the nanobacteria is at least partially broken down, dissolved, or removed by one or more of the dispersant and/or dissolution agents.

One class of deactivating agents is the various strong acids such as hydrochloric acid or sulfuric acid provided that a pH of the acid solution is generally low enough to attack the bacteria and thus generally has a pH of about 6.0 or less and desirably about 3 or less.

Strong bases can also be utilized and examples include sodium hydroxide, potassium hydroxide, ammonium hydroxide, sodium carbonate, and the like, which have a pH of about 9 or greater and desirably at least about 12 or greater.

Another class of suitable deactivation agents includes various aldehydes such as formaldehyde, glutaraldehyde, ortho-phthaldehyde, or formaldehyde-releasing agents such as hexamethylenetetramine, triazines, imidazoles, or hydantions and combinations thereof.

Alkylating agents include ethylene oxide, propylene oxide and the like.

Still another class of deactivating agents is phenols including substituted phenols such as cresols and bisphenols. Examples include alkyl and dialkyl phenols; dihydric phenols such as catechol, resorcinol, and hydroquinone; alkyl dihydroxybenzenes; halogen substituted phenols, such as chlorophenols, alkyl and/or aromatic substituted chlorophenols; nitrophenols, dinitrophenols, trinitrophenols, and alkyl or aromatic substituted nitrophenols; aminophenols; aromatic, alkyl aromatic, and aromatic alkyl substituted phenols; hydroxybenzoic acids; bisphenols, bis(hydroxyphenyl) alkanes, and hydroxyquinolines such as 8-hydroxyquinoline, and -combinations thereof. Desired phenolic compounds include o-phenylphenol (OPP), p-t-amylphenol (PTAP), o-benzyl-p-chlorophenol (OBPCP), p-chloro-m-xylenol (PCMX), 5-chloro-2-(2,4-dichlorophenoxy)phenol (Triclosan), and combinations thereof.

Aliphatic alcohols containing from 1 to about 20 carbon atoms can also be utilized such as ethanol, isopropanol, benzyl alcohol, methanol, and the like, with 3 to about 7 carbon atoms being desired such as butyl alcohol and various isomers thereof, hexanol alcohol, and heptanol alcohol. Various 5-carbon atom alcohols are highly desired such as n-pentyl alcohol, isopentyl alcohol, neopentyl alcohol, and cyclopentyl alcohol.

Halogen and halogen-releasing compounds constitute another class of deactivating agents and include iodine, iodophors such as PVPI, chlorine, hypochlorous acid and hypochlorites, chloramines, chlorine dioxide, chlorine donors such as sodium dichloroisocyanurate, bromine, hypobromous acid and hyprobromites, bromine-releasing compounds such as Bronopol®, and combinations thereof.

An important class of deactivating agents is various peroxygens and other forms of oxygen including peracids such as peracetic acid, perchromic acid, persulfuric acid, perbenzoic acid, organic or inorganic peroxides such as hydrogen peroxide, percarbonic acid, permanganate, perlauric acid, perglutaric acid, Magnesium peroxyphthalate, and combinations thereof. The use of hydrogen peroxide is optionally and hence may not be utilized.

Other compounds include oxidizing agents such as ozone in the form of either a gas, a vapor, or dissolved in a liquid such as water, or radicals such as hydroxyl, hydroperoxyl, superoxide, and oxide, and the like. Still other deactivating agents include nitrogen compounds such as urea, guanidine hydrochloride, and the like.

Yet other deactivating agents are various essential oil disinfection formulations that include various components of thyme oil, oregano oil, orange oil, lemon oil, tea tree oil, pine oil such as alpha-terpineol, non-polar hydrocarbons having a total of from about 6 to about 20 carbon atoms such as various aliphatics, aromatics, or combinations thereof with specific examples including hexane, octane, decane, benzyene, toluene, xylene, etc.; and various nitrogenous compounds such as methylenebisthiocyanate, DBNPA, pyridines, thiazoles, imidazoles, quinolines, anilides; various nitro compounds; and cocotrimethylenediamine; and docecylmorpholine-N-oxide; and polymerics such as biguanides and ionenes.

While the various deactivating agents can be in the form of a liquid, vapor, or a gas, or a combination thereof, generally liquids are desired.

These deactivating blends can then be mixed with the above dispersants such as anionic wetting agents and subsequently used to disinfect or sterilize various articles contaminated with nanobacteria. The nanobacteria deactivation compositions of the present invention can be applied by various methods with a one-step or two-step application generally being utilized. In the one-step application, a suitable class and amount of the deactivation agent are blended with one or more dispersants and/or one or more dissolution agents. The composition is then applied to the article to be treated. In a two or three step method, optionally the surface of the article is pretreated with various cleaners or physical treatments such as acidic cleaners, detergents or soaps, and the like, with physical treatment including hot water, steam, high temperatures such as autoclaving, radiation such as ultraviolet, or cleaning gas compounds such as ozone. When acid cleaning is utilized, usually the pH of the solution is below 6 and warm solutions are desired. Also, inhibiting agents such as those noted below are utilized to reduce corrosion of the metal article such as steel, copper, and the like. Subsequent to treatment, the surface is treated with one or more dispersants and/or dissolution agents. After the protective layer has been broken down, then the deactivation agent is applied to attack the nanobacteria.

The amount of the one or more deactivating agents will depend upon various factors such as the amount and concentration of nanobacteria, the strength and effectiveness of the deactivating agents, physical phase of the treatment, pH of the treatment, presence of additional components in the formulation, and the like.

In order to prevent the various deactivating agents such as strong acids and bases from attacking the metal of the articles being treated by the compositions of the present invention, corrosion inhibitors are utilized. Such compounds are well known to the art and to the literature and examples thereof include thiourea, ammonium thiocyanate, orthophosphate, polyphosphate, hydroxyphosphonoacetic acid, molybdate, zinc, amines, imidazolines, and the like. Inhibitors can also be produced by a Mannich condensation reaction utilizing formaldehyde, various amines and ketones. For copper or copper alloy articles, corrosion inhibitors such as tolyltriazole or benzotriazole can be utilized.

Suitable amounts of the various types of deactivating agents and the various dispersants and/or dissolution agents are utilized so that the nanobacteria deactivation compositions utilized to treat various articles achieve a nanobacteria log reduction of generally at least 4, i.e. disinfection, desirably at least 6, i.e. sterilization, and preferably at least 7. Generally suitable amounts range from about 5 to about 100,000 or 50,000 or 10,000 parts by weight and desirably from about 200 to about 1,000 parts by weight of the nanobacteria dissolution composition.

The present invention will be better understood by reference to the following proposed examples which serve to illustrate, but not to limit the present invention.

Proposed Formulation 1

5-7.5% HCl (sulfuric acid could be substituted) (deactivating agent)
0.25-0.75% ammonium bifluoride (utilized if silica is present to remove the same)
0.2-0.3% thiourea or propargyl alcohol (corrosion inhibitor)
0.03% nonionic wetting agent such as an alkylarylpolyethoxy alcohol (dispersant)
   Proposed Formulation 2
3-10% peracetic acid (deactivating agent)
0-10% nonionic surfactant such as a nonyl phenol ethoxylate (dispersant)
1-10% thiourea (corrosion inhibitor)
   Proposed Formulation 3
3-10% peracetic acid (deactivating agent)
2% polymaleic acid (dispersant)
   Proposed Formulation 4
10% DETA phosphonate (dissolution agent)
5% Belsperse 164 (dispersant)
5% polymaleic acid (dispersant)
0.1-10% Chlorine dioxide (deactivating agent)

As apparent from the above formulations wherein the percents are amounts by weight, numerous combinations of one or more dispersants and/or dissolution agents can be utilized in association with one or more deactivating agents to achieve disinfection or sterilization of an article surface.

While in accordance with the patent statutes, the best mode and preferred embodiment have been set forth, the scope of the invention is not limited thereto, but rather by the scope of the attached claims.

What is claimed is:

1. A nanobacteria deactivation composition, comprising:
a dispersant comprising a hydrophilic polymeric dispersant; or an anionic wetting agent, a nonionic wetting agent, or a cationic wetting agent, or an amphoteric wetting agent, or combinations thereof, said anionic wetting agent being free of a sodium organo sulfate, and a sodium aliphatic-aryl sulfonate, and said cationic wetting agent being free of a quaternary aliphatic-aryl ammonium chloride; and
a deactivating agent.

2. A nanobacteria deactivation composition, comprising:
a dissolution agent comprising a nitrogen-free organic acid having at least one carboxylic acid group and a total of from 2 to about 20 carbon atoms, a phosphoric acid containing compound, a sulfonated polyphosphoric acid compound, a polyphosphonate having three or more phosphonate groups, an enzyme; or salts thereof; or combinations thereof; said dissolution agent being free of an organic acid having from three to about five carboxylic acid groups; and
a deactivating agent

3. A nanobacteria deactivation composition according to claim 2, including a dispersant comprising a hydrophilic polymeric dispersant; or an anionic wetting agent, a nonionic wetting agent, or a cationic wetting agent, or an amphoteric wetting agent, or combinations thereof, said anionic wetting agent being free of a sodium organo sulfate, and a sodium aliphatic-aryl sulfonate, and said cationic wetting agent being free of a quaternary aliphatic-aryl ammonium chloride.

4. A nanobacteria deactivation composition, comprising:
a nonionic dispersant and a sterilizing alcohol having from 1 to about 20 carbon atoms, or
said sterilizing alcohol with a nanobacteria deactivating agent.

5. A nanobacteria deactivation composition according to claim 4, wherein said sterilizing alcohol contains approximately 5 carbon atoms.
